(19) United States
(12) Patent Application Publication
(10) Pub. No.: US 2018/0092779 A1
(43) Pub. Date: Apr. 5, 2018

(54) EASY TO APPLY DRESSINGS

(71) Applicant: Avery Dennison Corporation, Glendale, CA (US)

(72) Inventors: Sandy THEUNISSEN, Vosselaar (BE); Anne Marie WIBAUX, Fontainebleau (FR); Colleen WARD, Vosselaar (BE)

(21) Appl. No.: 15/567,783

(22) PCT Filed: Apr. 25, 2016
(86) PCT No.: PCT/US16/29166

§ 371 Date: Oct. 19, 2017

Related U.S. Application Data
(60) Provisional application No. 62/152,344, filed on Apr. 24, 2015.

(51) Int. Cl. A61F 13/02

(52) U.S. Cl. CPC A61F 13/024; A61F 13/023; A61F 13/0263

(57) ABSTRACT

A medical dressing having a multilayer assembly is described. The dressing includes a polyurethane film layer that is adhesively secured over a wound or other location on a user's skin. Also described are label strips that can be incorporated into the dressing. The label strips can include text, indicia, logos, or other markings which are applied to the strip prior to or after application of the dressing. A perforated single panel liner is utilized which covers the adhesive face of the film layer. The single panel liner includes a line of perforations extending across the liner.

### CROSS-REFERENCE TO RELATED APPLICATION

The present application claims the benefit of U.S. Provisional Application No. 62/152,344 filed on Apr. 24, 2015, which is incorporated herein by reference in its entirety.

### FIELD

The present subject matter relates to medical dressings, and particularly those made from films which are oxygen permeable and have high moisture vapor permeability but which are impermeable to liquid water and bacteria. The subject matter also relates to methods of producing such dressings and similar products.

### BACKGROUND

Wound dressings made with composite layers of film and adhesive which have high moisture vapor transmission rates are well known in the art. These dressings are used in many applications and are beneficial in that they are impervious to bacteria and liquid water but yet allow oxygen to penetrate the dressing from the ambient atmosphere and allow moisture from the skin of the patient to escape from beneath the dressing.

The dressings are typically provided with a release sheet covering the adhesive surface of the dressing. The release sheet is removed from the dressing when the dressing is applied to the patient. The thinness of the film and its flexibility allows the film to turn over onto itself during attempts to apply the film dressing to a patient. When a portion of the adhesive surface of the film touches other portions of the adhesive surface, the film dressing sticks to itself and makes it extremely difficult to separate the adhered portions and then apply to the patient.

In order to overcome this problem, film dressings of this type have been provided with adhesive-free tabs at opposite ends of the film. In some products there is a reinforcing member at the tab ends to provide a grasping or holding surface to be used to apply the dressings to a patient. Still other products utilize a support frame for the film.

Although satisfactory in certain aspects, incorporating tabs, reinforcing members, and/or support frames into such dressings increases manufacturing complexity and cost. In addition, depending upon the configuration of the dressing, it may be difficult and/or tedious to release the dressing film from the support frame and the release sheet during application.

Accordingly, in view of these and other objectives, a need exists for a dressing or thin film article which can easily be applied while avoiding contact with the adhesive face of the film.

### SUMMARY

The difficulties and drawbacks associated with previously known dressings are addressed in the present articles and related methods.

In one aspect, the present subject matter provides a dressing assembly comprising a polymeric film defining an inner face, an oppositely directed outer face, and a peripheral edge. The dressing also comprises adhesive disposed on the inner face of the film. The dressing may optionally comprise a label strip disposed on and secured to the film. And, the dressing also comprises a removable and perforated single panel liner disposed on the adhesive to thereby protect the adhesive until removal of the perforated single panel liner. The perforated single panel liner defines at least one line of perforation(s).

As will be realized, the subject matter described herein is capable of other and different embodiments and its several details are capable of modifications in various respects, all without departing from the claimed subject matter. Accordingly, the drawings and description are to be regarded as illustrative and not restrictive.

### BRIEF DESCRIPTION OF THE DRAWINGS

FIG. 1 is a perspective, schematic exploded assembly view of a dressing having a perforated single panel liner, dressing film, and a label strip in accordance with the present subject matter.

FIG. 2 is a schematic cross section of end regions of the dressing in FIG. 1, illustrating the perforated single panel liner, dressing film, and label strip in greater detail.

FIG. 3 is a schematic view of an embodiment of a perforated single panel liner in accordance with the present subject matter.

FIG. 4 is a schematic view of another embodiment of a perforated single panel liner in accordance with the present subject matter.

FIG. 5 is a schematic view of another embodiment of a perforated single panel liner in accordance with the present subject matter.

FIG. 6 is a schematic view of another embodiment of a perforated single panel liner in accordance with the present subject matter.

FIG. 7 is a schematic view of another embodiment of a perforated single panel liner in accordance with the present subject matter.

FIG. 8 is a schematic view of another embodiment of a perforated single panel liner in accordance with the present subject matter.

FIG. 9 is a schematic view of end regions of another dressing utilizing the liner depicted in FIG. 8.

### DETAILED DESCRIPTION OF THE EMBODIMENTS

The present subject matter provides dressings which include a thin polymeric film, a perforated single panel liner, and one or more label or date strips. The label strip can include text, indicia, logos, or other markings. Specifically, the dressings generally comprise (i) a thin dressing film, (ii) one or more adhesives on a face of the dressing film, (iii) a perforated single panel liner covering the one or more adhesives prior to use or application of the dressing, and (iv) one or more optional label strips incorporated in the dressing and typically disposed on an outer face of the dressing film. The dressings may additionally include one or more optional support layers, frames, and/or other components.

Details as to each of the components of the dressings are as follows.

### Dressing Film

In many embodiments of the present subject matter, the dressing film is made of a thin, flexible, conformable, resilient, supple, limp or flimsy material that can flex or bend to conform to irregular surfaces or contours, such as those of anatomical body parts. Typically, dressing film is sufficiently resilient to stretch or flex in response to movement or flexing of the dressing liner and/or support film and to conform to the user. The dressing film is typically transparent or substantially transparent to permit visualization through the dressing film. Alternatively, the dressing film can be opaque. The dressing film can be air permeable to allow oxygen to penetrate the dressing as well as moisture vapor permeable to allow moisture from the skin surface to escape through the dressing, and the dressing film can be liquid, air and bacteria impermeable. Non limiting examples of materials suitable for the dressing film include polymeric materials, such as polyurethane, polyesters and various copolyesters, elastomeric polyester, polyethylene, blends of polyurethane and polyester, chlorinated polyethylene, styrene/butadiene block copolymers and polyvinyl chloride, formed into continuous films or sheets by casting, extrusion or other processes. The dressing film can have various sizes and configurations dependent on the surfaces to which the dressing is to be applied. The dressing film may have a square or rectangular peripheral configuration with rounded or radiused corners. The present subject matter includes nearly any shape for the dressing film. In many embodiments, the dressing film has a minimal thickness, typically in the range of 0.0005 inch to 0.004 inch, and particularly about 0.0015 inch, with conformability of the dressing increasing with decreasing thickness of the dressing film. The dressing film has a maximum thickness of 0.04 inch to 0.118 inch.

### Adhesives

Adhesive is provided on a skin contacting side or face of the dressing film to cover at least the entire area circumscribed by the final dressing portion and, in many embodiments for ease of manufacturing, adhesive covers the entire area of face of the dressing film including a peripheral backing portion. Adhesive can be provided on a face of the dressing film in many various ways including solvent spreading, spraying, coating and extrusion, for example, and the adhesive can be applied directly to the dressing film or indirectly via a carrier sheet. The adhesive utilized is typically a pressure sensitive, skin contact adhesive that is hypo-allergenic and non-irritating to skin. Representative non-limiting examples of materials suitable for the adhesive include acrylate copolymers, such as copolymers of 2-ethylhexylacrylate and vinyl acetate with or without a cross-linking agent, water based adhesives and hot melt adhesives, for example. If desired, various medicaments and/or antimicrobial agents can be included in the adhesive to promote healing and inhibit infection. The adhesive is typically applied on a face of the dressing film in a thin layer, such as on the order of 0.001 inch to 0.010 inch in thickness or greater (according to the desired application), sufficient to obtain adequate skin adhesion without impairing the air and moisture vapor transmission characteristics of the dressing. In many embodiments, the adhesive is an acrylic adhesive.

### Perforated Single Panel Liner

Various materials can be utilized for the perforated single panel liner including conventional smooth surface paper materials, polyester films and polyolefin films of the type typically utilized as release liners, such as, for example, kraft paper, polyethylene and particularly high density polyethylene (HDPE), polypropylene, polyester and combinations and/or composites thereof. In certain applications, the perforated single panel liner is sufficiently thick, i.e., on the order of 0.004 inch to 0.0075 inch in thickness or higher, to rigidify a backing sheet if used, prior to use. The perforated single panel liner is typically sufficiently rigid and/or thick to typically maintain a flat configuration. However, the perforated single panel liner can have some flexibility to bend, flex or deform in response to external pressure. Depending on the material utilized for the perforated single panel liner, the surface can be coated with a release agent, such as fluorochemicals or silicone, for example, to facilitate release of the liner portion from the adhesive of the final dressing. It will be appreciated that the material utilized for the perforated single panel liner, with or without a release agent, can be varied to achieve a desired bond or tenacity of adhesion between the perforated single panel liner and the adhesive and that the bond or tenacity of adhesion can be selected such that manual removal or peeling away of the perforated single panel liner portion from the adhesive face of the thin film can be accomplished with a gentle pressure or pulling force and without damage to or impairment of the dressing.

The perforated single panel liner includes at least one perforation or “line of perforation(s)” as referred to herein extending across a span of the liner, from one edge to another edge or at least substantially so. The present subject matter includes an assortment of patterns, arrangements, and layouts for the line of perforation(s). Generally, the line of perforations includes at least one perforation or severed segment extending through the thickness of the liner. The present subject matter includes embodiments in which the perforations or a portion of the perforations do not extend through the entire thickness of the liner. In one embodiment, the perforation does not extend to more than 1/10 to more than 99/100 the thickness of the liner, preferably from 2/10 to 75/100, 2/10 to 50/100 of the thickness of the liner. The line of perforations may optionally include at least one tie region or “tie” which is free of any perforation(s) or severed segments. The perforation(s) and optional ties generally extend along a line and in many embodiments, along a straight line. However, it will be appreciated that the present subject matter includes line(s) of perforations which are not straight and instead are arcuate, nonlinear, jagged, or some other shape. The line of perforation can be in any location on the liner. In a preferred embodiment, the line of perforation is positioned between 5 mm from one edge border (66, 68, 70 and 72 in figures) to the middle of the dressing. In another preferred embodiment, the line of perforation is position between 5 mm from one side edge border (66 or 68) to the middle of the dressing. In many embodiments, the line of perforations includes a plurality of perforations each having the same length or substantially so. And, in many embodiments, the line of perforations includes a plurality of ties each having the same length or substantially so. In still other embodiments, the line of perforations includes a plurality of perforations in which at least two of the perforations have different lengths. And, certain embodiments include the line of perforations having at least two of the ties with different lengths. For embodiments in which the lengths of perforations within a line of perforations are different, typically the shorter length perforations are located proximate a central location or region of the line of perforations. In still other embodiments in which the lengths of ties within a line of perforations are different, typically the shorter length ties are located proximate a central location or region of the line of perforations. In particular embodiments, the total length of all ties along a line of perforations is less than 75%, less than 60%, less than 50%, less than 40%, less than 30%, less than 20%, less than 10%, based upon the length of the line of perforations. Representative and nonlimiting examples of lengths for perforations are within a range of from about 2 mm to about 30 mm, and lengths for ties are within a range of from about 0.2 mm to about 2 mm. Particular combinations of tie and perforation lengths are as follows: (i) 0.2 mm, 30 mm; (ii) 0.2 mm, 2 mm; (iii) 2 mm, 30 mm; and (iv) 2 mm, 2 mm. However, it will be understood that the present subject matter includes a wide array of other lengths and combinations of lengths, and in no way is limited to these particular representative values. In certain embodiments, the line of perforation formed in the perforated single panel line is configured to “break” or split apart along the line at a level of force that is less than the release force between the liner and the adhesive face of the dressing. In particular embodiments, the break force is at least 20 g/25 mm (length of the line of perforations) less than the release force. Additional aspects of the line of perforations are provided herein.

### Optional Label Strips

As previously noted, an optional label strip or date strip can be incorporated into the dressings of the present subject matter and can include text, indicia, logos, or other markings. In certain embodiments of the present subject matter, the label strip is attached to an outer face of the transparent film. Attachment can be accomplished by a variety of techniques, however, attachment is typically achieved by adhesive. After application of the dressing to skin, the label strip remains attached to the outer face of the transparent film. Prior to (or after) application, writing or other operations can be performed to provide text or information on the label strip, which will remain with the dressing even after application.

A wide range of materials can be used for the label strips that are associated with the dressings. In certain embodiments, the label strips are formed from paper or paper based materials. However, the present subject matter includes forming the label strips from polymeric materials. It is also contemplated that the label strips can be formed from combinations of paper and polymeric materials and be in the form of composite materials. The present subject matter also includes forming the label strips with one or more regions or layers of paper material(s) and one or more regions or layers of polymeric materials. Furthermore, one or both faces of the label strip and particularly a print-receiving face can be coated with a print-receptive topcoat to promote ink adherence or ink “wet-out.” Moreover, one or both faces of the label strip could be subjected to a surface treatment operation such as corona treatment or plasma treatment.

The label strip can also be coated with a release coating so that an adhesive coated portion of an optional support layer can extend over the label strip. This configuration provides extra support to the dressing on application and before removal of the support layer. In this case the release coating is chosen to prevent permanent or strong adhesion of the support layer.

The one or more label strip(s) are attached to the dressing film and typically disposed on an outer face of the dressing film. The label strip(s) can be secured to the dressing film such as by adhesive. The label strip(s) in certain versions of the dressings, are provided immediately alongside a recessed side edge of the dressing and/or an optional support layer. However, it will be understood that the present subject matter includes a wide range of arrangements, configurations, and variations of the dressings described herein.

In certain embodiments of the present subject matter, the label strip may include one or more lines of perforation. These lines of perforation in the label strip include at least one perforation and may optionally include one or more ties. The characteristics of the lines of perforation in the label strip are the same or at least similar to those of the lines of perforation in the single panel release liner, and so the description of the lines of perforation in the perforated single panel liner apply.

### Optional Support Layer

As noted, the dressings of the present subject matter can include one or more optional support layers. Transparent or translucent film materials are particularly intended as supporting layers. However, opaque or non-transparent paper or film materials can be used alternatively. Useful as supporting layers are particularly those films that are manufactured from polyester, polyethylene, polypropylene, polyvinylchloride, polystyrene, polyamide, polycarbonate, cellulose ester, ethylene vinyl acetate, polyvinyl acetate, polyvinyl alcohol and/or combinations thereof. Particularly useful are supporting layers from polyester or polyethylene or polypropylene. The present subject matter also includes the use of paper or paper-based materials for the support component. Combinations of polymeric and paper materials can also be used. In certain embodiments, the thickness of the supporting layers are adjusted to a thickness of about 1 to about 80 μm, 10 to 75 μm particularly from about 20 to about 60 μm and especially from about 20 to about 40 μm.

In a particular embodiment of the current invention, the support layer is disposed over the line of perforation of the panel liner. In a preferred embodiment of the current invention, the support layer is disposed on the outer face of the perforated release liner. The support layer may cover the entire perforation or a portion of the perforation. In one embodiment, the support layer covers at least 50% of the perforation. In another embodiment, the support layer covers 100% of the perforation.

### Representative Embodiments

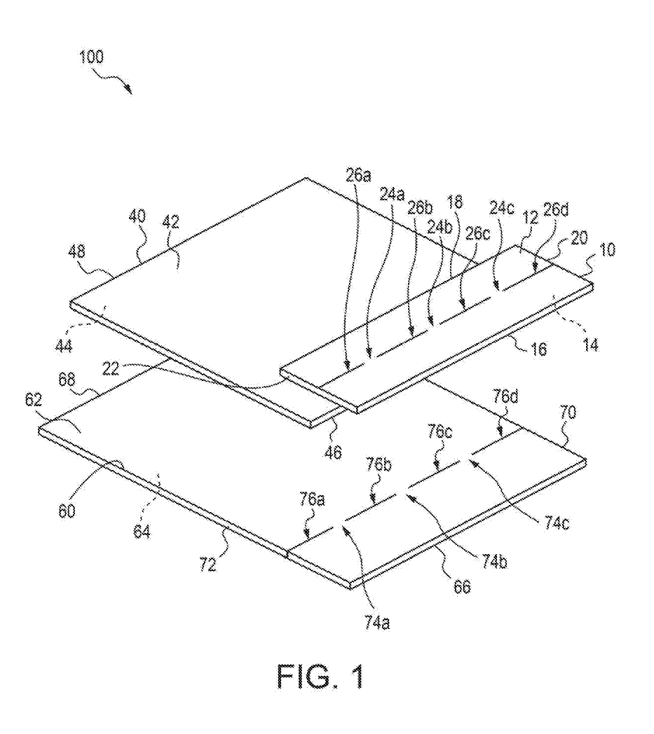

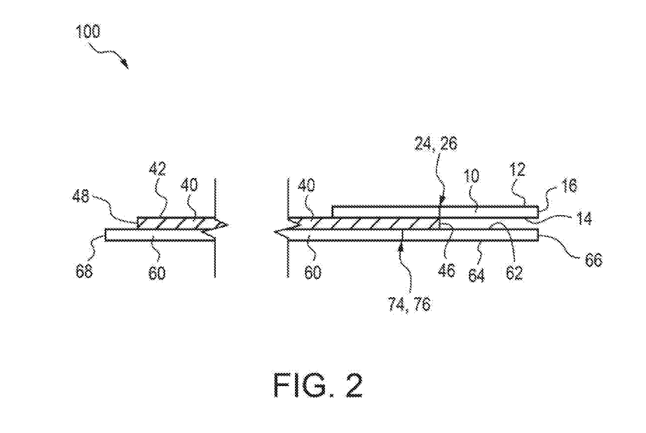

FIGS. 1 and 2 illustrate a dressing 100 in accordance with the present subject matter. The dressing 100 comprises a label strip 10, an adhesive coated dressing film 40, and a perforated single panel release liner 60. The label strip 10 defines an outer face 12 and an oppositely directed inner face 14. The outer face 12 may receive text, markings, and/or indicia as described herein. The label strip faces 12, 14 extend between edges 16, 18, 20, and 22. The label strip 10 is disposed on an outer face 42 of the dressing film 40. In the particular version of dressing 100 shown, the label strip 10 is oriented parallel to and immediately alongside a recessed edge 46 of the dressing film 40. A thin layer or coating of adhesive resides on an inner face 44 of the dressing film 40. The perforated single panel release liner 60 covers the adhesive, and specifically a release coated face 62 of the liner 60 contacts the adhesive layer on the inner face 44 of the dressing film 40. The perforated single panel release liner 60 also defines an outer face 64 which is opposite the release coated face 62.

In the particular version of the dressing 100 depicted in FIGS. 1 and 2, a side edge 66 or peripheral edge portion of the perforated single panel liner 60 extends outward beyond the corresponding edge 46 of the dressing film 40, and in certain versions to the same extent as a corresponding edge 16 of the label strip 10, as depicted in FIG. 2. And, in the particular version of the dressing 100 shown in FIGS. 1 and 2, a side edge 68 or peripheral edge portion of the perforated single panel liner 60 extends outward beyond a corresponding edge 48 of the dressing film 40.

FIG. 2 is a schematic cross sectional view of two opposite edge portions of the dressing 100 depicted in FIG. 1. In the particular embodiment of dressing 100, both of the label strip 10 and the perforated single panel liner 60 extend beyond the edge 46 of the film 40. And, at the opposite edge of the dressing 100, the perforated single panel liner 60 extends beyond the edge 48 of the film 40. It will be understood that the present subject matter is not limited to this particular configuration. Instead, the present subject matter includes a variety of different arrangements and configurations for the various layers and components of the dressings.

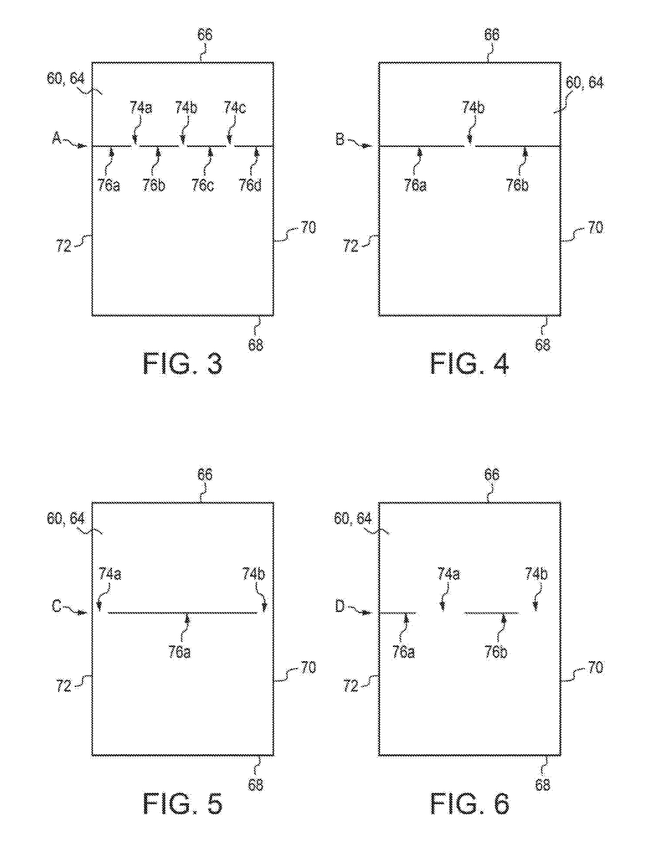

As previously noted, the present subject matter dressings utilize a perforated single panel release liner. The perforated single panel liner includes at least one line of perforation(s) that extends across a span of the liner, from one edge to another edge, or at least substantially so. FIGS. 3-8 illustrate various representative examples of perforated single panel liners that include at least one line of perforations. Referring to FIG. 3, an outer face 64 of release liner 60 from the dressing 100 of FIGS. 1-2 is depicted. The release liner 60 defines a plurality of edges 66, 68, 70, and 72 that extend around the liner 60. The liner 60 includes at least one line of perforations collectively shown as A in FIG. 3. The line A extends between edges 70 and 72. The line of perforations A includes an alternating collection of perforations 76 and ties 74 each of which extend along or substantially so, the line A. In the particular embodiment shown in FIG. 3, the line of perforations includes perforations 76a, 76b, 76c, and 76d; and ties 74a, 74b, and 74c. FIG. 1 also illustrates these perforations and ties in the perforated single panel liner 60. It will be appreciated that the present subject matter includes a wide array of different configurations for the line of perforations. Generally, the line of perforations can include a number of perforations such as from 1 to 50, more particularly from 1 to 25, more particularly from 1 to 10, and in certain versions, from 1 to 5. The line of perforations can include a number of ties such as from 0 to 50, more particularly from 1 to 25, more particularly from 1 to 10, and in certain versions, from 1 to 5. In many embodiments, the line of perforations in the single panel liner which can include ties and perforations 74, 76 respectively; underlies a location between opposite edges 46 and 48 of the film 40. This configuration is shown in FIG. 2.

FIG. 4 depicts another version of the liner 60 having a line of perforations B extending between the edges 70 and 72. The line of perforations B includes perforations 76a and 76b, and a single tie 74b.

FIG. 5 depicts another version of the liner 60 having a line of perforations C extending between the edges 70 and 72. The line of perforations C includes a single perforation 76a and ties 74a and 74b.

FIG. 6 depicts another version of the liner 60 having a line of perforations D extending between the edges 70 and 72. The line of perforations D includes perforations 76a and 76b, and ties 74a and 74b.

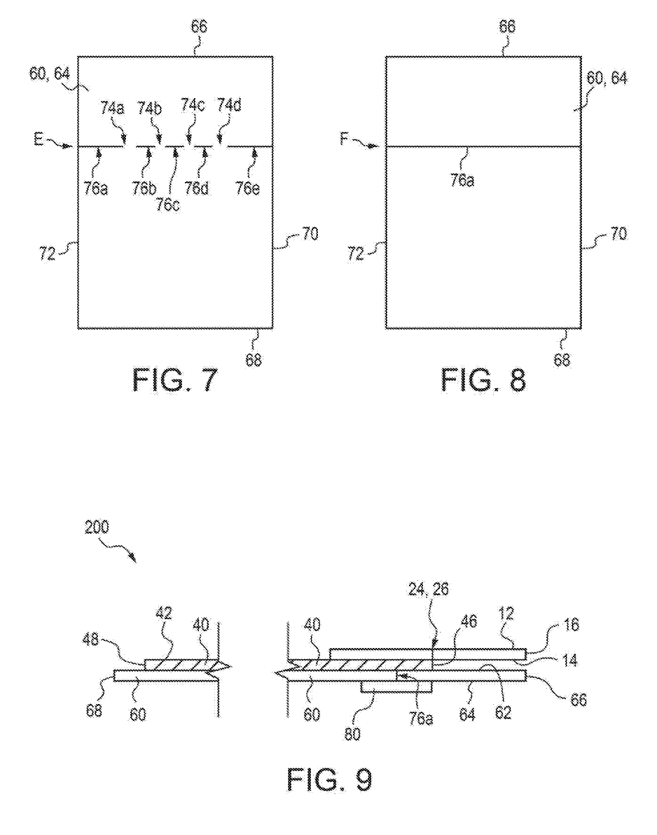

FIG. 7 depicts another version of the liner 60 having a line of perforations E extending between the edges 70 and 72. The line of perforations E includes a perforations 76a, 76b, 76c, 76d, and 76e, and ties 74a, 74b, 74c, and 74d.

FIG. 8 depicts another version of the liner 60 having a line of perforations F extending between the edges 70 and 72. The line of perforations F includes a single perforation 76a and no ties.

FIG. 9 is a schematic cross section of end regions of another dressing 200 utilizing the perforated single panel liner 60 of FIG. 8. In this version of the present subject matter, a support component 80 can optionally be applied to cover or overlay the line of perforations F. The use of the support component 80 may be beneficial to impart additional rigidity to the dressing 200. Additionally, the use of support component 80 serves to create a stop or break when removing the liner from the adhesive face while reinforcing the perforation(s). This configuration provides a stronger bond between the liner components. Furthermore, the use of support component 80 provides protection from environmental contaminants such as gases or moisture.

In certain embodiments of the present subject matter, the label strip can include one or more line(s) of perforations. These line(s) of perforations are as previously described with regard to the line(s) of perforations of the perforated single panel release liner. Referring to FIGS. 1 and 2, the line(s) of perforations typically extend between opposite edges 20, 22 of the label strip 10. If one or more line(s) of perforations are provided in a label strip, typically they include one or more perforations 26 such as shown in FIG. 1 as perforations 26a, 26b, 26c, and 26d; and one or more ties 24 such as shown in FIG. 1 as ties 24a, 24b, and 24c.

In particular embodiments, the line of perforations in the label strip which can include ties and perforations 24, 26, respectively; overlies the edge 46 of the film 40 as shown in FIG. 2.

The perforation(s) in the single panel liner and/or in the label strip can be formed using a wide range of techniques including conventional cutting and stamping operations known in the art. In many embodiments, the perforations are formed in the single panel liner prior to application with the dressing film. However, the present subject matter includes forming the perforations in the single panel liner after application or contact with the dressing.

Many other benefits will no doubt become apparent from future application and development of this technology. For example, the label strip can add additional bulk to the limp flimsy film to facilitate removal of the dressing from the skin after use. Due to the low caliper of the dressings it is typically very difficult to get a starting point for removal. The extra thickness and stiffness at the label strip position can significantly assist or promote removal efforts. The dressings of the present subject matter are particularly useful as incise film dressings and as donor site dressings.

All patents, published applications, and articles noted herein are hereby incorporated by reference in their entirety.

As described hereinabove, the present subject matter solves many problems associated with previous strategies, systems and/or devices. However, it will be appreciated that various changes in the details, materials and arrangements of components, which have been herein described and illustrated in order to explain the nature of the present subject matter, may be made by those skilled in the art without departing from the principle and scope of the claimed subject matter, as expressed in the appended claims.

1. A dressing assembly comprising:
a polymeric film defining an inner face, an oppositely directed outer face, and a peripheral edge;
adhesive disposed on the inner face of the film;
a removable single panel liner disposed on the adhesive to thereby protect the adhesive until removal of the single panel liner, the single panel liner defining at least one line of perforation(s).

2. The dressing of claim 1 further comprises:
a label strip disposed on and secured to the film.

3. The dressing of claim 1 wherein the line of perforation(s) extends entirely across the single panel liner.

4. The dressing of claim 1 wherein the line of perforation(s) includes at least one tie and at least one perforation.

5. The dressing of claim 1 wherein the line of perforations includes a number of perforations within a range of from 1 to 50.

6. The dressing of claim 5 wherein the number of perforations is within a range of from 1 to 25.

7. The dressing of claim 6 wherein the number of perforations is within a range of from 1 to 10.

8. (canceled)

9. The dressing of claim 1 wherein the line of perforations includes a number of ties within a range of from 0 to 50.

10. The dressing of claim 9 wherein the number of ties is within a range of from 1 to 25.

11. (canceled)

12. (canceled)

13. The dressing of claim 1 wherein the polymeric film includes polyurethane.

14. The dressing of claim 1 wherein the adhesive is an acrylic adhesive.

15. The dressing of claim 2 wherein the label strip defines at least one line of perforation(s).

16. The dressing of claim 15 wherein the line of perforation(s) extends entirely across the label strip.

17. The dressing of claim 15 wherein the line of perforation(s) includes at least one tie and at least one perforation.

18. The dressing of claim 15 wherein the line of perforations includes a number of perforations within a range of from 1 to 50.

19. The dressing of claim 18 wherein the number of perforations is within a range of from 1 to 25.

20. (canceled)

21. (canceled)

22. The dressing of claim 15 wherein the line of perforations includes a number of ties within a range of from 0 to 50.

23. The dressing of claim 22 wherein the number of ties is within a range of from 1 to 25.

24. (canceled)

25. (canceled)

26. A dressing assembly comprising:
a polymeric film defining an inner face, an oppositely directed outer face, and a peripheral edge;
adhesive disposed on the inner face of the film;
a removable single panel liner disposed on the adhesive to thereby protect the adhesive until removal of the single panel liner, the single panel liner defining at least one line of perforation(s);
wherein the removable single panel liner has an outer face opposite the adhesive;
a support layer disposed on the outer face of the removable single panel liner covering at least a portion of the at least one line of perforation(s).

27. The dressing assembly of claim 26, wherein the support layer covers at least 50% of the at least one line of perforation(s).

28. (canceled)
